(19) United States
(12) Patent Application Publication
(10) Pub. No.: US 2026/0283979 A1
(43) Pub. Date: Sep. 24, 2026

(54) PHARMACEUTICAL COMPOSITION COMPRISING UBIQUINONE-5 AND METHOD OF USE THEREOF

(71) Applicant: The Trustees of Columbia University in the City of New York, New York, NY (US)

(72) Inventor: Richard J. LEVY, New York, NY (US)

(73) Assignee: The Trustees of Columbia University in the City of New York, New York, NY (US)

(21) Appl. No.: 19/692,095

(22) Filed: May 29, 2026

Related U.S. Application Data
(63) Continuation of application No. 18/463,787, filed on Sep. 8, 2023, now abandoned.
Application No. 18/463,787 is a continuation of application No. PCT/US2022/019895, filed on Mar. 11, 2022.
(60) Provisional application No. 63/159,755, filed on Mar. 11, 2021.

(51) Int. Cl. A61K 31/122; A61K 9/00; A61K 9/107; A61K 47/22; A61P 23/00

(52) U.S. Cl. CPC A61K 31/122; A61K 9/0019; A61K 9/107; A61K 47/22; A61P 23/00

(57) ABSTRACT

The present disclosure provides for a pharmaceutical composition comprising ubiquinone-5 (UB5) and method of use thereof. The present disclosure relates to ubiquinone-5 used as an anesthetic, sedative agent, hypnotic agent, or combination thereof.

### CROSS-REFERENCE TO RELATED APPLICATIONS

This application is a continuation application of U.S. patent application Ser. No. 18/463,787 filed on Sep. 8, 2023, which is a continuation application of PCT/US2022/019895 filed on Mar. 11, 2022, which claims priority to U.S. Provisional Patent Application No. 63/159,755 filed on Mar. 11, 2021, the content of each of which is incorporated herein by reference in its entirety.

### GOVERNMENT LICENSE RIGHTS

This invention was made with government support under NS112706 awarded by the National Institutes of Health. The government has certain rights in the invention.

### BACKGROUND

Anesthetics and sedative-hypnotic agents render subjects unconscious and insensitive to noxious stimulation. Modern day surgery and invasive therapy would not be possible without the advent of these unique compounds. Although sedative-hypnotics are widely used in clinical practice, the mechanisms by which they induce unconsciousness, amnesia, and immobilization are poorly understood. Furthermore, sedative-hypnotic agents cause undesirable adverse effects. Thus, there is a need to discover new sedative-hypnotics and to advance our knowledge of their mechanism(s) of action.

The field of anesthesiology is actively and continuously seeking new and novel anesthetic compounds. The purpose is to find the most efficacious agents with the greatest safety profile. Furthermore, identification of new agents will permit potential insights into mechanisms of action of all anesthetics (given that it is unknown how anesthetics induce unconsciousness in a reversible manner).

### SUMMARY

The present disclosure provides for a pharmaceutical composition comprising ubiquinone-5 (UB5). The present disclosure relates to ubiquinone-5 used as an anesthetic, sedative agent, hypnotic agent, or combination thereof.

Ubiquinone-5 induces mitochondrial proton leak, demonstrating favorable anesthetic, sedative, hypnotic properties in mice. Therefore, ubiquinone-5, can be an effective anesthetic with a favorable safety profile for clinical use.

In some embodiments, the pharmaceutical composition comprising ubiquinone-5 is an oil-in-water emulsion. In some embodiments, the composition is sterile. In some embodiments, the ubiquinone-5 is stabilized by means of a surfactant. In some embodiments, the ubiquinone-5 is dissolved in a water-immiscible solvent. In some embodiments, the ubiquinone-5 is emulsified with water. In some embodiments, the composition comprises an excipient selected from the group consisting of amino acids, vitamins, minerals, and a combination thereof. In some embodiments, the ubiquinone-5 is present in an amount from about 0.01% (w/v) to 5% (w/v). In some embodiments, the pH of the composition ranges from about 5.0 to about 8.0. In some embodiments, the composition comprises a tonicity modifier.

In some embodiments, the composition is for administration by a route selected from the group consisting of intravenous, inhalational, subcutaneous, intramuscular, transdermal, and parenteral administration. In some embodiments, the composition is administered to the subject an effective amount of a composition comprising a ubiquinone-5 containing oil-in-water emulsion. In some embodiments, the subject is a mammal. In some embodiments, the composition is administered at a dose sufficient to achieve a desired anesthetic endpoint, wherein the desired anesthetic endpoint is selected from the group consisting of general anesthesia, moderate sedation, tranquilization, immobility, amnesia, analgesia, deep sedation and autonomic quiescence. In some embodiments, the composition is administered by a route selected from the group consisting of intravenous, inhalational, subcutaneous, intramuscular, and transdermal.

In some embodiments, the composition is prepared by dispersing at least one surfactant; dissolving ubiquinone-5 in at least one water-immiscible solvent to form a non-aqueous ubiquinone-5 solution; and adding the non-aqueous ubiquinone-5 solution to the surfactant dispersion to form a crude oil-in-water emulsion of ubiquinone-5. In some embodiments, the crude oil-in-water emulsion of ubiquinone-5 is sterilized to obtain a sterile oil-in-water emulsion of ubiquinone-5.

In some embodiments, the ubiquinone-5 is dissolved in an ethanol/intralipid solvent.

In some embodiments, the pharmaceutical composition comprises: about 2% (w/v) ubiquinone-5; about 5% (v/v) ethanol; about 95% (v/v) intralipid. The intralipid can comprise about 20% soybean oil; about 2.25% (v/v) glycerin; about 1.2% (v/v) egg-yolk phospholipid; and about 76.55% (v/v) water.

In some embodiments, the pharmaceutical composition comprises: about 2% (w/v) ubiquinone-5; about 5% (v/v) ethanol; about 19% (v/v) soybean oil; about 2.1375% (v/v) glycerin; about 1.14% (v/v) egg-yolk phospholipid; and about 72.7225% (v/v) water.

In some embodiments, the present disclosure provides for a pharmaceutical composition comprising a ubiquinone-5 and decylubiquinone containing oil-in-water emulsion. In some embodiments, the ratio by weight of ubiquinone-5 to decylubiquinone is 1:1, 2:1, 3:1, 4:1, 5:1, 1:2, 1:3, 1:4, or 1:5.

In some embodiments, the present disclosure provides for a pharmaceutical composition comprising a ubiquinone-5 and a quinone analog containing oil-in-water emulsion, wherein the quinone analog is known to be anticardiotoxic or antiarrhythmic.

In some embodiments, the present disclosure provides for a method of inducing anesthesia in a subject, comprising administering to the subject an effective amount of a composition comprising a ubiquinone-5 containing oil-in-water emulsion, wherein the composition is administered at a dose of up to 200 mg/kg every 1 hour, at a dose of 80 to 200 mg/kg every 1 hour or at a dose of 20 to 200 mg/kg every 1 hour.

In some embodiments, the present disclosure provides for a method of sedating a patient in an intensive care unit, which comprises administering to the patient an effective amount of ubiquinone-5 or a pharmaceutically acceptable salt thereof, wherein the patient remains arousable and orientated.

In some embodiments, the present disclosure provides for a method of conducting a procedure involving sedation in a subject comprising administering intravenously to the subject one or more fixed doses of a pharmaceutical composition in an amount sufficient to sedate the subject to induce moderate anesthesia, wherein the pharmaceutical composition comprises ubiquinone-5 or a pharmaceutically acceptable salt thereof. In some embodiments, the procedure is minimally invasive such as endoscopy, cardiac catheterization, and interventional radiology procedures. In some embodiments, the procedure is noninvasive such as imaging procedures like MRI, CT scan, and XRadiation therapy.

In some embodiments, the present disclosure provides for a method of conducting a procedure involving sedation in a subject comprising administering intravenously to the subject one or more fixed doses of a pharmaceutical composition in an amount sufficient to sedate the subject to induce deep anesthesia, wherein the pharmaceutical composition comprises ubiquinone-5 or a pharmaceutically acceptable salt thereof, and passing an endoscope into the subject. In some embodiments, the procedure is invasive such procedures in operative environments or in the ICU.

### BRIEF DESCRIPTION OF THE DRAWINGS

The process of the disclosure will be described in more detail below and with reference to the attached drawings in which the same number is used for the same or similar elements, and where:

FIGS. 1a-c are representative tracings of complex II-dependent state 4 respiration (leak respiration) using succinate in the presence of oligomycin are depicted above and tracings of simultaneously measured mitochondrial membrane potential (A) are shown below, for (a) propofol, (b) decylubiquinone, and (c) ubiquinone-5;

FIGS. 2a and 2b shows ECG signals of murine hearts after exposure to (a) decylubiquinone and (b) uniquinone-5;

FIGS. 3a-c show the reaction of a mouse (a) during injection of ubiquinone-5 (b) immediately after injection and (c) at a time 3-4 minutes after injection;

FIG. 4 is a dose-response curve for 50 C57Bl/6 mice that were injected with various doses of ubiquinone-5;

FIG. 5 is a graph showing the latency of RORR in mice that were injected with various doses of ubiquinone-5;

FIG. 6a shows the ambulation of mice that were injected with various doses of ubiquinone-5;

FIG. 6b shows the ambulation of mice that were injected with ubiquinone-5 and a vehicle injection pre- and post-injection;

FIGS. 7a-b shows the EEG signals at various times for mice that were injected with (a) ubiquinone-5 and (b) intralipid vehicle;

FIG. 8a shows a time line for recordation of data for neural activity of mice injected with ubiquinone-5;

FIG. 8b shows the neuronal GCaMP6 (genetically encoded calcium indicator) fluorescence prior to injection (baseline) and 2 minutes post-injection;

FIG. 8c shows the fluorescent traces of Ca²⁺ transients from 60-100 neurons over time;

FIG. 8d shows the mean Ca²⁺ fluorescence over time;

FIG. 9 shows the concentration of ubiquinone-5 in mice brains as a function of dose;

FIG. 10a shows the O₂ consumption as a function of time for injections of GABA and ubiquinone-5; and

FIG. 10b shows the ΔΨm measurement as a function of time for injections of GABA and ubiquinone-5.

### DETAILED DESCRIPTION

Disclosed herein are pharmaceutical compositions comprising ubiquinone-5, methods of using said compositions, and method of making said compositions.

Existing anesthetics are associated with several side effects, and many approved anesthetics present risks for patients with cardiovascular disorders or other conditions. Therefore, there is a need to identify additional anesthetic compounds with more clearly defined mechanisms to minimize off target effects.

Propofol is considered to be a near-ideal anesthetic agent due to its rapid onset, short duration of action, and minimal side effects. Mahmoud et al., Recent advances in intravenous anesthesia and anesthetics, FlO00Res., 2018 April 17; 7. A critical feature of propofol and other anesthetics is their action on the electron transport chain (ETC), a cellular respiratory pathway found in mitochondria. Kelz et al., The biology of general anesthesia from paramecium to primate. Curr. Biol. 2019 Nov. 18; 29 (22): pp. Rl 199-R1210. However, propofol has some disadvantages such as increased risk of bacterial contamination and hyperlipidemia, as well as a potentially fatal risk of propofol infusion syndrome. Additionally, propofol is not suitable for long term administration, for example, in ICU patients. Hemphill S et al. Propofol Infusion Syndrome: A Structured Literature Review and Analysis of Published Case Reports. BJA 2019, 122:448-459.

Research has been focused on developing improved formulations and alternatives that overcome some of these disadvantages.

Coenzyme Q (also known as ubiquinone) is an electron carrier within the mitochondrial electron transport chain that is critical for cellular energy production (ATP). Quinones are vitamin-like lipid molecules that are analogues of the endogenous coenzyme Q molecule. Quinone analogues are classically used with in vitro experimentation to test and measure mitochondrial function.

In the description that follows, the term “inhalational anesthetic” refers to gases or vapors that possess anesthetic qualities that are administered by breathing through an anesthesia mask or ET tube connected to an anesthetic machine. Exemplary inhalational anesthetics include without limitation volatile anesthetics (halothane, isoflurane, sevoflurane and desflurane) and the gases (ethylene, nitrous oxide and xenon).

The term “injectable anesthetic or sedative drug” refers to anesthetics or sedatives that can be injected, e.g., under the skin, into a vein, etc. The injection may be via a hypodermic needle and syringe. Through actions on nerves in the brain or spinal cord, the anesthetic or sedative can either render an individual insensible to painful stimuli, or decrease an individual's perceived sensation of painful stimuli, or induce within an individual an amnestic and/or calming effect.

The term “effective amount” or “pharmaceutically effective amount” refer to the amount and/or dosage, and/or dosage regime of one or more compounds necessary to bring about the desired result e.g., an amount sufficient to effect anesthesia, render the subject unconscious and/or immobilize the subject.

The term “pharmaceutically acceptable” refers to a material, such as a carrier or diluent, which does not abrogate the biological activity or properties of the compound useful within the present disclosure, and is relatively non-toxic, i.e., the material may be administered to an individual without causing undesirable biological effects or interacting in a deleterious manner with any of the components of the composition in which it is contained.

The term “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

The term “composition” or “pharmaceutical composition” refers to a mixture of at least one compound useful within the present disclosure with a pharmaceutically acceptable carrier. The pharmaceutical composition facilitates administration of the compound to a subject.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The terms “patient,” “individual,” “subject” interchangeably refer to any mammal, e.g., a human or non-human mammal, e.g., a non-human primate, a domesticated mammal (e.g., canine, feline), an agricultural mammal (e.g., equine, bovine, ovine, porcine), or a laboratory mammal (e.g., rattus, murine, lagomorpha, hamster).

The term “molar water solubility” refers to the calculated or measured number of moles per liter of a compound present at a saturated concentration in pure water at 25° C. and at pH=7.0.

The term “intensive care unit” encompasses any setting that provides intensive care.

The term “sedation” refers to a relaxed, calm state of the body and mind which is induced pharmacologically, e.g., by the use of sedatives. This also encompasses “analgosedation” which includes the concomitant application of an analgesic drug. Furthermore, as defined herein, the term sedation includes also deep sedation, preoperative sedation, anxiolysis, and amnestic use for perioperative events, conscious sedation during short diagnostic, operative or endoscopic procedures, and sedation prior and/or concomitant to the administration of other anesthetic or analgesic agents.

The phrase “treated or administered in combination” as used herein for the combined therapeutic use or administration of ubiquinone-5 means that at least one dose of ubiquinone-5 is given within a time frame, where the substance exhibits a pharmacological effect. The ubiquinone-5 may be administered concomitantly or sequentially. This phrase encompasses treatments in which ubiquinone-5 is administered either by the same route or different routes of administration.

The term “analgesia” as used herein refers to the pharmacologically induced absence or deadening of the sense of pain, e.g., by the use of analgesics, such as opioids.

The term “fixed dose” as used in the present disclosure relates to an amount of a drug given to a patient irrespective of his body weight.

As used herein the term “initial dose” is synonymous to the term “loading dose” and is defined as the first dose of a drug given in the context of a medical sedative treatment.

The term “minimal sedation” or “mild sedation” refers to a drug-induced state during which the patient responds normally to verbal commands. Cognitive function and coordination may be impaired. Ventilatory and cardiovascular functions are unaffected. Minimal sedation is also known as anxiolysis.

The term “moderate sedation” (synonymously with conscious sedation) refers to a drug-induced depression of consciousness during which the patient responds purposefully to verbal command, either alone or accompanied by light tactile stimulation. No interventions are necessary to maintain a patent airway. During moderate sedation spontaneous ventilation is adequate and the cardiovascular function is usually maintained.

The term “deep sedation” refers to a drug-induced depression of consciousness during which the patient cannot be easily aroused but responds purposefully following repeated or painful stimulation. Independent ventilatory function may be impaired. The patient may require assistance to maintain a patent airway. During deep sedation the spontaneous ventilation may be inadequate and cardiovascular function is usually maintained.

The term “procedural sedation” refers to a technique of administering sedatives or dissociative agents with or without analgesics to induce a state that allows the patient to tolerate unpleasant procedures while maintaining cardio-respiratory function. Procedural sedation and analgesia is intended to result in a depressed level of consciousness that allows the patient to maintain oxygenation and airway control independently.

The term “analgosedation” refers to a pharmacologically induced analgesia with concurrent sedation. In contrast to the anesthesia the patient can react on external stimuli and breathe unaided. Dependent on the dose of the sedative and/or the analgesic drug the analgosedation can, intentionally or not, reach the state of general anesthesia.

The term “general anesthesia” refers to a drug-induced loss of consciousness (LoC) during which the patient is not arousable, even to painful stimuli. During general anesthesia the ability to maintain independent ventilatory function is often impaired and assistance is often required in maintaining a patent airway. Furthermore, positive pressure ventilation may be required due to depressed spontaneous ventilation or drug-induced depression of neuromuscular function and cardiovascular function may be impaired.

For assessment of the various states of sedation and analgosedation the so-called Modified Observer's Assessment of Alertness and Sedation scale (MOAA/S) (Nonaka, Takashi et. al., 2018, Can sedation using a combination of propofol and dexmedetomidine enhance the satisfaction of the endoscopist in endoscopic submucosal dissection? Endoscopy International Open. 06. E3-E10. 10.1055/s-0043-122228) and, alternatively, the Ramsey Scale (Rasheed A M, et. al, A. Ramsay Sedation Scale and Richmond Agitation Sedation Scale: A Cross-sectional Study. Dimens Crit Care Nurs. 2019 March/April; 38 (2): 90-95, doi: 10.1097/DCC.0000000000000346. PMID: 30702478) often are used. These scales are as follows:

TABLE 1
Modified Observer's Assessment of Alertness/Sedation Scale

| Responsiveness | Score |
|---|---|
| Agitated | 6 |
| Responds readily to name spoken in normal tone (alert) | 5 |
| Lethargic response to name spoken in normal tone | 4 |
| Responds only after name is called loudly and/or repeatedly | 3 |
| Responds only after mild prodding or shaking | 2 |
| Does not respond to mild prodding or shaking | 1 |
| Does not respond to deep stimulus | 0 |

Ramsey Sedation cafe

| Responsiveness | Score |
|---|---|
| Patient is anxious and agitated or restless, or both | 1 |
| Patient is cooperative, oriented and tranquil | 2 |
| Patient responds to commands only | 3 |
| Patient exhibits brisk response to light glabellar tap or | 4 |
| loud auditory stimulus | |
| Patient exhibits a sluggish response to light glabellar tap or | 5 |
| loud auditory stimulus | |
| Patient exhibits no response | 6 |

The term “opioid” which is synonymous to the term “opioid drug” as used herein refers to compounds which have the same mode of action as the constituents of opium, the dried milky liquid of the poppy seed, Papaver somniferum.

The term “amnestic use” as used herein relates to the induction of amnesia, which represents the partial or total loss of memory.

The term “operative procedure” as used herein refers to all kind of medical intervention into the living body, either invasive or non-invasive, for diagnostic and/or therapeutic purposes. Medical intervention in particular comprises medical treatments which, on a regular basis, are expected to cause post-operative pain for the patient. As a synonymous term for “operative procedure” the term “surgery” is also used herein.

Manual or mechanical ventilation is defined as external assistance in breathing by manual or mechanical methods such as e.g., mask ventilation, or intubation.

As used herein, the term “solvate” refers to a complex of variable stoichiometry formed by a solute (in this present disclosure, a compound of formula (I), an opioid or a salt thereof) and a solvent. Such solvents for the purpose of the present disclosure may not interfere with the biological activity of the solute. Examples of suitable solvents include, but are not limited to water, methanol, ethanol and acetic acid. The solvent used can be a pharmaceutically acceptable solvent. Examples of suitable pharmaceutically acceptable solvents include water, ethanol and acetic acid.

It should be noted that when “about” is at the beginning of a numerical list, “about” modifies each number of the numerical list. The term “about” in reference to a numeric value refers to +10% of the stated numeric value. In other words, the numeric value can be in a range of 90% of the stated value to 110% of the stated value.

Ubiquinone-5 is a member of the family of ubiquinones. It shares a quinine chemical group with other ubiquinones but differs in the number of isoprenyl chemical subunits in its tail. Ubiquinone-5 is not naturally occurring. The formula for ubiquinone-5 is 2,3-Dimethoxy-5-methyl-6-(3-methyl-2-butenyl)-1,4-benzoquinone. The chemical structure of ubiquinone-5 is shown below:

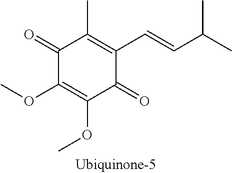 [SMILES: COC1=C(OC)C(=O)C(/C=C/C(C)C)=C(C)C1=O]

Coenzyme Q (CoQ) compounds are lipid soluble components of cell membranes. They perform multiple functions such as electron and proton transport. Coenzyme Ql0 (CoQl0) is a component of the electron transport chain and participates in aerobic cellular respiration, generating energy in the form of ATP. Crane, F. L. Biochemical functions of coenzyme Ql0. J. Am. Coll. Nutr. 20(6), 591-598 (2001).

Ubiquinone-5 (which is equivalent to CoQl) is an amphipathic CoQl0 homolog that has a tail consisting of one isoprene unit. It has been used as an electron acceptor to study a range of oxidoreductases as isolated enzymes, in subcellular fractions, in intact cells in culture, and in perfused organs. Bongard et al. Coenzyme Q(l) as a probe for mitochondrial complex I activity in the intact perfused hyperoxia-exposed wild-type and Nqol-null mouse lung. American Journal of Physiology Lung Cellular and Molecular Physiology 302(9), L949-L958 (2012).

Ubiquinone analogs, including ubiquinone-5, impact mitochondrial permeability transition pore (PTP) formation, as well as PTP-dependent cell death, in an analog- and cell-specific manner. Devun et al. Ubiquinone analogs: A mitochondrial permeability transition pore-dependent pathway to selective cell death. PLOS One 5(7), (2010).

The ubiquinone-5 containing compositions of the present disclosure are useful as anesthetics including sedation, and induction and maintenance of general anesthesia. Thus, in another aspect, the present disclosure provides a method for inducing anesthesia in mammals which comprises parenteral administration of a sterile, aqueous pharmaceutical composition comprising a ubiquinone-5.

The composition of the present disclosure may comprise about 0.01% (w/v) to about 5% (w/v) of ubiquinone-5 or from about 0.05% (w/v) and about 2% (w/v) of ubiquinone-5. The composition may be sterile.

The ubiquinone-5 may be dissolved in a pharmaceutically acceptable water-immiscible solvent and emulsified in water and said emulsion stabilized by means of a surfactant; or the ubiquinone-5 may itself be emulsified in water without addition of a water-immiscible solvent and said emulsion stabilized by means of a surfactant.

Water-immiscible solvents suitable for the preparation of oil-in-water emulsions suitable for parenteral administration are known to those skilled in the pharmaceutical arts (Handbook of Pharmaceutical Excipients Wade and Weller, Eds. (1994), American Pharmaceutical Association, The Pharmaceutical Press: London, pp 451-453). Typically, the water-immiscible solvent can be a vegetable oil, for example, soybean oil, safflower oil, cottonseed oil, corn oil, sunflower oil, arachis oil, castor oil and combinations thereof. The water-immiscible solvent can also be a mono-, di-, and triglycerides, fatty acid esters, or chemically modified vegetable oils, physically modified vegetable oils and combinations thereof. The present disclosure may also comprise any combination of said water-immiscible solvents. When used, the water-insoluble solvent comprises up to about 30% weight of the composition, in the range of about 5% to about 25% weight, in the range of about 10% to about 20% weight, or about 10% weight.

The composition of the present disclosure comprises a pharmaceutically acceptable surfactant which aids in the emulsification of the water-immiscible phase in water and stabilizes said emulsion. Suitable surfactants include naturally occurring surfactants: for example, egg or soy phosphatides, either in their native or modified forms; non-ionic surfactants, for example a polyethylene glycol or esters thereof, NP-40 or Triton X-100; or any mixture thereof. In some embodiments, the surfactant is egg-yolk phospholipid. The amount of surfactant effective in producing and maintaining a stable oil-in-water emulsion will depend on the particular formulation. The factors and their relationships are well known to skilled practitioners in the pharmaceutical arts. These factors include the presence or absence of a water-immiscible solvent, the particular water-immiscible solvent used, the particular surfactant employed, the presence of salts, and the pH of the composition.

The composition of the present disclosure is formulated with pH in the range of about 5.0 to about 8.0. The pH may be adjusted as required by means of addition of an alkali, for example sodium hydroxide, or an acid, for example hydrochloric acid.

The composition of the present disclosure may be made isotonic with blood by incorporation of a suitable tonicity modifier, for example glycerin, dextrose, mannitol, or sodium chloride.

The composition of the present disclosure may include an excipient selected from the group consisting of amino acids, vitamins, minerals, and a combination thereof.

The compositions of the present disclosure are sterile, aqueous formulations and are prepared by standard manufacturing techniques using, for example, aseptic manufacturing methods and sterilization by autoclaving.

In some embodiments, the ubiquinone-5 composition includes ascorbic acid or its pharmaceutically acceptable salt thereof. The pharmaceutically acceptable salt is selected from the group consisting of sodium ascorbate, potassium ascorbate, calcium ascorbate, magnesium ascorbate, and combinations thereof. In some embodiments, the composition comprises from about 0.05% (w/w) to about 0.2% (w/w), 0.05% (w/w) to about 0.1% (w/w), 0.05% (w/w), 0.2% (w/w) or 0.1% (w/w) of the ascorbic acid or its pharmaceutically acceptable salt.

In some embodiments, the ubiquinone-5 composition is for administration by a route selected from the group consisting of intravenous, inhalational, subcutaneous, intramuscular, transdermal, and parenteral administration.

In some embodiments, the pharmaceutical composition comprises: about 2% (w/v) ubiquinone-5; about 5% (v/v) ethanol; about 19% (v/v) soybean oil; about 2.1375% (v/v) glycerin; about 1.14% (v/v) egg-yolk phospholipid; and about 72.7225% (v/v) water.

The ubiquinone-5 containing compositions of the present disclosure are useful as anesthetics including sedation, and induction and maintenance of general anesthesia. Thus, in another aspect, the present disclosure provides a method for inducing anesthesia in mammals which comprises parenteral administration of a sterile, aqueous pharmaceutical composition comprising ubiquinone-5.

Dosage levels appropriate for the induction of desired degree of anesthesia, for example sedation, or induction of or maintenance of general anesthesia, by the compositions of the present disclosure will depend on the type of mammal under treatment and the physical characteristics of the specific mammal under consideration. These factors and their relationship in determining this amount are well known to skilled practitioners in the medical arts. Approximate dosage levels may be derived from the substantial literature, may be tailored to achieve optimal efficiency, and will be contingent on myriad factors recognized by those skilled in the medical arts including weight, diet, and concurrent medication.

Oil-in-water emulsion including total-parenteral-nutrition formulations are administered by infusion to patients for whom oral nutrition is impossible, undesirable, or insufficient. The emulsified lipids provide a concentrated caloric content. These formulations may also contain other nutrients, for example amino acids, vitamins, and minerals. Commercial examples of such formulations include INTRALIPID® (trademark Pharmacia), LIPOFUNDINO® (trademark Braun), and TRA V AMULSION® (trademark Baxter).

In some embodiments, the present disclosure is directed to a method of inducing anesthesia in a subject, comprising administering to the subject an effective amount of a composition comprising a ubiquinone-5 containing oil-in-water emulsion.

In some embodiments, the present disclosure is directed to a method of inducing anesthesia in a subject, comprising administering to the subject an effective amount of a composition comprising a sterile ubiquinone-5 containing oil-in-water emulsion.

The ubiquinone-5 containing composition may be administered as a single bolus or a continuous infusion.

In some embodiments, the single bolus injection of the composition can be in an amount dependent on the body mass of the subject ranges from about

$$5 - 500 mg / kg , 5 - 200 mg / kg , 5 - 150 mg / kg , 5 - 100 mg / kg , 5 - 75 mg / kg , 5 - 50 mg / kg , 5 - 20 mg / kg , 20 - 500 mg / kg , 20 - 200 mg / kg , 20 - 150 mg / kg , 20 - 100 mg / kg , 20 - 75 mg / kg , 20 - 50 mg / kg , 50 - 500 mg / kg , 50 - 200 mg / kg , 50 - 150 mg / kg , 50 - 100 mg / kg , 50 - 75 mg / kg , 75 - 500 mg / kg , 75 - 200 mg / kg , 75 - 150 mg / kg , 75 - 100 mg / kg , 100 - 500 mg / kg , 100 - 200 mg / kg , 100 - 150 mg / kg , 150 - 500 mg / kg , or 150 - 200 mg / kg .$$

In some embodiments, the single bolus injection of the composition in an amount not dependent on the body mass of the subject that is ranges from about

$$0.4 - 35000 mg , 0.4 - 14000 mg , 0.4 - 7000 mg , 0.4 - 3500 mg , 0.4 - 10 mg , 0.4 - 1 mg , 1 - 35000 mg , 1 - 14000 mg , 1 - 7000 mg , 1 - 3500 mg , 1 - 10 mg , 10 - 35000 mg , 10 - 14000 mg , 10 - 7000 mg , 10 - 3500 mg , 3500 - 35000 mg , 3500 - 14000 mg , 3500 - 7000 mg , 7000 - 35000 mg , 7000 - 14000 mg , or 14000 - 35000 mg .$$

In some embodiments, the method of administration is by a number of bolus injections ranges from 2 to 10.

In some embodiments, the method of administration is by continuous infusion. In some embodiments, the composition is administered at a rate ranging from about 1 mg/kg/hr to about 100 mg/kg/hr, 1 mg/kg/hr to 50 mg/kg/hr, 1 mg/kg/hr to 25 mg/kg/hr, 25 mg/kg/hr to 100 mg/kg/hr, 25 mg/kg/hr to 50 mg/kg/hr, or 50 mg/kg/hr to 100 mg/kg/hr.

In some embodiments, the subject is a mammal. In some embodiments, the mammal is selected from the group consisting of a dog, a cat, a cow, and a horse. In some embodiments, the subject is a human.

In some embodiments, the composition is administered at a dose sufficient to achieve a desired clinical anesthetic endpoint.

The clinical anesthetic endpoint can be achieved when there is insensitivity to noxious stimulation (also referred to as “general anesthesia”), immobility, amnesia, analgesia, unconsciousness, sedation, and/or autonomic quiescence.

In some embodiments, the desired clinical anesthetic endpoint is selected from the group consisting of, moderate sedation, and tranquilization.

In some embodiments, the dose ranges from about

$$5 - 500 mg / kg , 5 - 200 mg / kg , 5 - 150 mg / kg , 5 - 100 mg / kg , 5 - 75 mg / kg , 5 - 50 mg / kg , 5 - 20 mg / kg , 20 - 500 mg / kg , 20 - 200 mg / kg , 20 - 150 mg / kg , 20 - 100 mg / kg , 20 - 75 mg / kg , 20 - 50 mg / kg , 50 - 500 mg / kg , 50 - 200 mg / kg , 50 - 150 mg / kg , 50 - 100 mg / kg , 50 - 75 mg / kg , 75 - 500 mg / kg , 75 - 200 mg / kg , 75 - 150 mg / kg , 75 - 100 mg / kg , 100 - 500 mg / kg , 100 - 200 mg / kg , 100 - 150 mg / kg , 150 - 500 mg / kg , or 150 - 200 mg / kg .$$

In some embodiments, the composition is administered by any route sufficient to achieve a desired anesthetic effect. In some embodiments, the composition is administered by a route selected from the group consisting of intravenous, inhalational, subcutaneous, intramuscular, and transdermal.

In some embodiments, the composition is administered over a period of time ranging from
about 0.1 hours to about 350 hours, about 0.1 hours to 48 hours, about 0.1 hours to 24 hours,
about 0.5 hours to about 350 hours, about 0.5 hours to 48 hours, about 0.5 hours to 24 hours,
about 1 hours to about 350 hours, about 1 hours to 48 hours, about 1 hours to 24 hours,
about 2 hours to about 350 hours, about 2 hours to 48 hours, about 2 hours to 24 hours,
about 3 hours to about 350 hours, about 3 hours to 48 hours, about 3 hours to 24 hours,
about 6 hours to about 350 hours, about 6 hours to 48 hours, about 6 hours to 24 hours,
about 12 hours to about 350 hours, about 12 hours to 48 hours, or about 24 hours to about 350 hours.

In some embodiments, the composition is administered over a period of time ranging from
about 0.1 hours to about 12 hours, about 0.1 hours to about 6 hours, about 0.1 hours to about 3 hours,
about 0.5 hours to about 12 hours, about 0.5 hours to about 6 hours, about 0.5 hours to about 3 hours,
about 1 hours to about 12 hours, about 1 hours to about 6 hours, about 1 hours to 3 hours,
about 2 hours to about 12 hours, about 2 hours to about 6 hours, about 2 hours to 3 hours,
about 3 hours to about 12 hours, about 3 hours to about 6 hours,
about 6 hours to about 12 hours, about 12 hours to about 24 hours,
or about 24 hours to about 8 hours.

The composition can be administered prior to a medical procedure, during a medical procedure, or after a medical procedure.

In some embodiments, the pharmaceutical composition of ubiquinone-5 for parenteral administration, can be made by dispersing at least one surfactant; dissolving ubiquinone-5 in at least one water-immiscible solvent to form a non-aqueous ubiquinone-5 solution; and adding the non-aqueous ubiquinone-5 solution to the surfactant dispersion to form a crude oil-in-water emulsion of ubiquinone-5.

In some embodiments, the composition is made by further sterilizing the crude oil-in-water emulsion of ubiquinone-5 to obtain a sterile oil-in-water emulsion of ubiquinone-5.

In some embodiments, the surfactant can be selected from the group consisting of native egg phosphatide, modified egg phosphatide, native soy phosphatide, modified soy phosphatide, polyethylene glycol, polyethylene glycol esters and combinations thereof.

In some embodiments, the water-immiscible solvent is selected from the group consisting of soybean oil, safflower oil, cottonseed oil, corn oil, sunflower oil, arachis oil, castor oil, monoglycerides, diglycerides, triglycerides, fatty acid esters, chemically modified vegetable oils, physically modified vegetable oils and combinations thereof.

In some embodiments, the composition is made by further dissolving ascorbic acid or its pharmaceutically acceptable salts thereof in water to form an aqueous solution and adding the surfactant dispersion of step (a) to the aqueous solution to form a mixture, wherein the mixture is added to the non-aqueous ubiquinone-5 solution in step (c) to form the crude oil-in-water emulsion.

Another quinone analogue, decylubiquinone, causes mitochondrial proton leak and inhibits the electron transport chain in a discrete manner. Decylubiquinone is a member of the class of 1,4-benzoquinones that is 2,3-dimethoxybenzoquinone which has been substituted at positions 5 and 6 by decyl and methyl groups. Decylubiquinone has a role as a cofactor. It is derived from a 6-decylubiquinol and has the following structure:

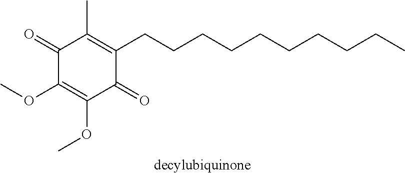 [SMILES: CCCCCCCCCCC1=C(C)C(=O)C(OC)=C(OC)C1=O]

Decylubiquinone is a known antagonist of ubiquinone-5. In some embodiments, an uniquinone-5 composition is mixed with decylubiquinone in order to offset certain negative biological impacts that high doses, such as about 200 mg/kg, of ubiquinone-5 can have on the heart.

In some embodiments, the present disclosure is directed to a pharmaceutical composition comprising a ubiquinone-5 and decylubiquinone containing oil-in-water emulsion. In some embodiments, the composition is sterile. In some embodiments, the ratio by weight of ubiquinone-5 to decylubiquinone is 1:1, 2:1, 3:1, 4:1, 5:1, 1:2, 1:3, 1:4, or 1:5. In some embodiments, the present disclosure is directed to method of inducing anesthesia in a subject, comprising administering to the subject an effective amount of a composition comprising a ubiquinone-5 and decylubiquinone containing oil-in-water emulsion. The ubiquinone-5 and decylubiquinone composition can be sterile.

In some embodiments, the present disclosure is directed to a process for preparing a pharmaceutical composition of ubiquinone-5 for parenteral administration, the process comprising: dispersing at least one surfactant; dissolving ubiquinone-5 and decylubiquinone in at least one water-immiscible solvent to form a non-aqueous ubiquinone-5 solution; and adding the non-aqueous ubiquinone-5 solution to the surfactant dispersion to form a crude oil-in-water emulsion of ubiquinone-5. In some embodiments, the process further comprises sterilizing the crude oil-in-water emulsion of ubiquinone-5 and decylubiquinone to obtain a sterile oil-in-water emulsion of ubiquinone-5 and decylubiquinone.

In some embodiments, the surfactant can be selected from the group consisting of native egg phosphatide, modified egg phosphatide, native soy phosphatide, modified soy phosphatide, polyethylene glycol, polyethylene glycol esters and combinations thereof.

In some embodiments, the water-immiscible solvent is selected from the group consisting of soybean oil, safflower oil, cottonseed oil, corn oil, sunflower oil, arachis oil, castor oil, monoglycerides, diglycerides, triglycerides, fatty acid esters, chemically modified vegetable oils, physically modified vegetable oils and combinations thereof.

In some embodiments, the ubiquinone-5 and decylubiquinone are dissolved in an ethanol/intralipid solvent.

In some embodiments, the pharmaceutical composition comprises: about 2% (w/v) ubiquinone-5; about 2% (w/v) decylubiquinone; about 5% (v/v) ethanol; about 95% (v/v) intralipid. The intralipid can comprise about 20% soybean oil; about 2.25% (v/v) glycerin; about 1.2% (v/v) egg-yolk phospholipid; and about 76.55% (v/v) water.

In some embodiments, the pharmaceutical composition comprises: about 2% (w/v) ubiquinone-5; about 2% (w/v) decylubiquinone; about 5% (v/v) ethanol; about 19% (v/v) soybean oil; about 2.1375% (v/v) glycerin; about 1.14% (v/v) egg-yolk phospholipid; and about 72.7225% (v/v) water.

In some embodiments, a ubiquinone-5 composition is mixed with other quinone analogues in order to offset certain negative biological impacts that high doses, such as about 200 mg/kg, of ubiquinone-5 can have on the heart. For example, ubiquinone-5 at high doses may cause bradycardia, heart block, asystole, and depressed ventricular function. These quinone analogues have anticardiotoxic and/or antiarrhythmic properties.

In some embodiments, the present disclosure is directed to a pharmaceutical composition comprising a ubiquinone-5 and a quinone analogue containing oil-in-water emulsion. In some embodiments, the composition is sterile. In some embodiments, the ratio by weight of ubiquinone-5 to quinone analogue is 1:1, 2:1, 3:1, 4:1, 5:1, 1:2, 1:3, 1:4, or 1:5. In some embodiments, the present disclosure is directed to method of inducing anesthesia in a subject, comprising administering to the subject an effective amount of a composition comprising a ubiquinone-5 and quinone analogue containing oil-in-water emulsion. The ubiquinone-5 and quinone analogue composition can be sterile.

In some embodiments, the present disclosure is directed to a process for preparing a pharmaceutical composition of ubiquinone-5 for parenteral administration, the process comprising: dispersing at least one surfactant; dissolving ubiquinone-5 and quinone analogue in at least one water-immiscible solvent to form a non-aqueous ubiquinone-5 solution; and adding the non-aqueous ubiquinone-5 solution to the surfactant dispersion to form a crude oil-in-water emulsion of ubiquinone-5.

In some embodiments, the ubiquinone-5 and quinone analogue are dissolved in an ethanol/intralipid solvent.

In some embodiments, the pharmaceutical composition comprises: about 2% (w/v) ubiquinone-5; about 2% (w/v) quinone analogue; about 5% (v/v) ethanol; about 95% (v/v) intralipid. The intralipid can comprise about 20% soybean oil; about 2.25% (v/v) glycerin; about 1.2% (v/v) egg-yolk phospholipid; and about 76.55% (v/v) water.

In some embodiments, the pharmaceutical composition comprises: about 2% (w/v) ubiquinone-5; about 2% (w/v) quinone analogue; about 5% (v/v) ethanol; about 19% (v/v) soybean oil; about 2.1375% (v/v) glycerin; about 1.14% (v/v) egg-yolk phospholipid; and about 72.7225% (v/v) water.

In some embodiments, the present disclosure is directed to a method of inducing anesthesia in a subject, comprising administering to the subject an effective amount of a composition comprising a ubiquinone-5 containing oil-in-water emulsion, wherein the composition is administered at a dose of up to 200 mg/kg every 1 hour, at a dose of 80 to 200 mg/kg every 1 hour or at a dose of 20 to 200 mg/kg every 1 hour.

In some embodiments, the present disclosure is directed to a method for sedating a patient in the ICU and encompasses potential ICU uses of ubiquinone-5 and a pharmaceutically acceptable salt thereof.

In some embodiments, the present disclosure is directed to a method for sedating a patient in the ICUs that are intubated. In some embodiments, the present disclosure is directed to a method for sedating a patient in the ICUs that are not intubated.

In some embodiments, the present disclosure is directed to a method for sedating a patient that are experiencing seizures, withdrawal, or combative patients that require invasive monitoring.

Patients recovering from an episode of critical illness have reported factors they found most distressing during their ICU stay (Gibbons, C. R., et al., Clin. Intensive Care 4 (1993) 222-225). The most consistently unpleasant memories are anxiety, pain, fatigue, weakness, thirst, the presence of various catheters, and minor procedures such as physiotherapy. The aim of ICU sedation is to ensure that the patient is comfortable, relaxed, and tolerates uncomfortable procedures such as placement of iv-lines or other catheters but is still arousable.

At the moment, there is no universally accepted sedative regimen for critically ill patients. Thus, these patients receive a variety of drugs during their stay in an ICU, often receiving the variety of drugs concurrently. The agents used most commonly are given to achieve patient comfort. Various drugs are administered to produce anxiolysis (benzodiazepines), amnesia (benzodiazepines), analgesia (opioids), antidepression (antidepressants/benzodiazepines), muscle relaxation, sleep (barbiturates, benzodiazepines, propofol) and anesthesia (propofol, barbiturates, volatile anesthetics) for unpleasant procedures. These agents are cumulatively called sedatives in the context of ICU sedation, though sedation also includes the treatment of conditions that affect patient comfort, such as pain and anxiety, and many of the drugs mentioned above are not considered sedatives outside the context of ICU sedation.

The presently available sedative agents are associated with such adverse effects as prolonged sedation or oversedation (propofol and especially poor metabolizers of midazolam), prolonged weaning (midazolam), respiratory depression (benzodiazepines, propofol, and opioids), hypotension (propofol bolus dosing), bradycardia, ileus or decreased gastrointestinal motility (opioids), immunosuppression (volatile anesthetics and nitrous oxide), renal function impairment, hepatotoxicity (barbiturates), tolerance (midazolam, propofol), hyperlipidemia (propofol), increased infections (propofol), lack of orientation and cooperation (midazolam, opioids, and propofol), and potential abuse (midazolam, opioids, and propofol).

In addition to the adverse effects of every individual sedative agent, the combination of these agents (polypharmacy) may cause adverse effects. For example, the agents may act synergistically, which is not predictable; the toxicity of the agents may be additive; and the pharmacokinetics of each agent may be altered in an unpredictable fashion. In addition, the possibility of allergic reactions increases with the use of more than one agent. Furthermore, these adverse effects might necessitate the use of additional agents to treat the adverse effects, and the additional agents themselves may have adverse effects.

Patients will be sedated by ubiquinone-5 or a pharmaceutically acceptable salt thereof and will be arousable and oriented, which makes the treatment of the patient easier. The patients will be awakened and they will be able to respond to questions. They will be aware, but not anxious, and tolerate an endotracheal tube well. Should a deeper level of sedation or more sedation be required or desired, an increase in ubiquinone-5 dose will smoothly transits the patient into a deeper level of sedation. Ubiquinone-5 would not have adverse effects associated with other sedative agents, such as, respiratory depression, nausea, prolonged sedation, ileus or decreased gastrointestinal motility, or immunosuppression. Lack of respiratory depression would allow ubiquinone-5 to be used also for non-ventilated, critically ill patients who require sedation, anxiolysis, analgesia, and hemodynamic stability yet must remain oriented and easily aroused. A predictable pharmacological response will be achieved by administering ubiquinone-5 or a pharmaceutically acceptable salt thereof to a patient in the ICU.

Ubiquinone-5 or a pharmaceutically acceptable salt thereof will be administered perorally, transmucosally, transdermally, intravenously or intramuscularly. One skilled in the art would recognize the doses and dosage forms suitable in the method of the present disclosure. The precise amount of the drug that will be administered according to the present disclosure will be dependent on numerous factors, such as the general condition of the patient, the condition to be treated, the desired duration of use, the route of administration, the type of mammal, etc. The dose range of ubiquinone-5 can be described as target plasma concentrations. The plasma concentration range anticipated to provide sedation in the patient population in the ICU varies between 0.1-2 ng/ml depending on the desired level of sedation and the general condition of the patient. These plasma concentrations can be achieved by intravenous administration by using a bolus dose and continuing it by a steady maintenance infusion. For example, in some embodiments, the dose range for the bolus to achieve the forementioned plasma concentration range in a human will be about 0.2-2 μg/kg, about 0.5-2 μg/kg, or 1.0 μg/kg, to be administered in about 10 minutes or slower, followed by a maintenance dose of about 0.1-2.0 μg/kg/h, about 0.2-0.7 μg/kg/h, or about 0.4-0.7 μg/kg/h. The time period for administering ubiquinone-5 or a pharmaceutically acceptable salt thereof depends on the desired duration of use.

In some embodiments, tissue levels in the brain of ubiquinone-5 after a single bolus will be in the range of from about 10 μM to 50 μM.

In some embodiments, the present disclosure is directed to a method of sedating a patient in an intensive care unit, which comprises administering to the patient an effective amount of ubiquinone-5 or a pharmaceutically acceptable salt thereof, wherein the patient remains arousable and orientated. In some embodiments, the present disclosure is directed to a method of sedating a patient in an intensive care unit, which comprises administering to the patient an effective amount of ubiquinone-5 or a pharmaceutically acceptable salt thereof, wherein the patient is unarousable and not orientated. The unarousable state of the patient could be due to an illness that the patient has or if the patient is purposefully induced into a medically induced coma.

In some embodiments, the present disclosure is directed to using an effective amount of ubiquinone-5 or a pharmaceutically acceptable salt thereof as a replacement for situations where propofol would have otherwise been used.

In some embodiments, the ubiquinone-5 or pharmaceutically acceptable salt is the sole active agent.

In some embodiments, the present disclosure is directed to a method of sedating a patient in an intensive care unit, comprising administering a pharmaceutical composition to the patient, wherein the pharmaceutical composition comprises an active agent and an inactive agent, wherein the active agent consists of ubiquinone-5 or a pharmaceutically acceptable salt thereof, and wherein the patient remains arousable and orientated.

In some embodiments, the ubiquinone-5 or pharmaceutically acceptable salt is administered intravenously. In some embodiments, a loading dose and a maintenance dose of ubiquinone-5 are administered. In some embodiments, the patient is a human. In some embodiments, the loading dose of ubiquinone-5 is 2 mg/kg to 200 mg/kg. In some embodiments, the loading dose is administered in about 10 minutes. In some embodiments, the maintenance dose of ubiquinone-5 is 1 mg/kg/h to 100 mg/kg/hr.

In some embodiments, ubiquinone-5 or a pharmaceutically acceptable salt or solvate thereof will be used for induction of sedation.

In some embodiments, ubiquinone-5 will be administered in combination with an opioid. In some embodiments, both drugs will be given intravenously. Moreover, both drugs can be administered in a fixed dose. The dose of ubiquinone-5 can vary between about 2 to about 1000 mg, between about 3 mg and about 900 mg, or between about 5 and about 800 mg. In a particular embodiment of the present disclosure 10 mg ubiquinone-5 is administered.

In one embodiment, ubiquinone-5 will be administered as a bolus injection with a concentration of ubiquinone-5 in the range of from 1 mg/ml to 20 mg/ml.

In an embodiment at least one further top-up dose of ubiquinone-5 will follow the initial dose of ubiquinone-5. The top-up dose will be between about 1.5 mg to about 14000 mg, about 1.5 mg to about 1400 mg, about 1.5 mg to about 3 mg, about 3 mg to about 1400 mg, or about 1400 mg to about 14000 mg.

In some embodiments, ubiquinone-5 will be administered in a fixed dose, whereas the initial dose and the top-up doses are combined as follows:
8 mg initial dose plus 2 or 3 mg top-up dose,
7 mg initial dose plus 2 or 3 mg top-up dose,
5 mg initial dose plus 2 or 3 mg top-up dose,
4 mg initial dose plus 2 or 3 mg top-up dose, or
3 mg initial dose plus 2 or 3 mg top-up dose.

In a particular embodiment the initial dose and the top-up(s) will be selected to provide a maximum dose of 10 mg per treatment.

In a further aspect of the present disclosure, ubiquinone-5 will be administered in a fixed dose, whereas the initial dose and the top-up doses are combined as follows:
8 mg initial dose plus 3 mg top-up dose, or
7 mg initial dose plus 2 mg top-up dose, or
5 mg initial dose plus 3 mg top-up dose.

In an embodiment, the patients will receive an initial single intravenous dose of ubiquinone-5 over one minute. In another aspect of the present disclosure the ubiquinone-5 top-up dose will be administered not less than 2, in some embodiments 3-4 minutes apart from the starting or the previous top up dose. In one aspect of the present disclosure up to a maximum of six top-up doses of ubiquinone-5 will be given, so that not more than seven doses of ubiquinone-5 are given per treatment. In some embodiments, the number of top ups is below 3, or even below 2.

The medical treatment—and thus the required sedation according to the present disclosure—can last less than one hour, less than 45 minutes or less than 30 minutes.

According to the present disclosure the top-up doses can contain the identical or a different amount of ubiquinone-5.

In another aspect of the present disclosure the dosing regimen will be adjusted in order to maintain a MOAA/S score of less than or equal to than 4, a MOAA/S score of 1 to 4, or a MOAA/S score of 2 to 4. This adjustment can be performed by alteration of the top-up doses with regard to the dose of the top-up dose or the time interval between the top-up doses or both. In a further aspect of the present disclosure a change in the time interval between the top-up doses will be used for maintaining the level of the MOAA/S score. Hereby, the time interval will be shortened in case that the patient exhibits reduced sedation and prolonged in case of increased sedation.

In a further aspect of the present disclosure the dosing regimen will be adjusted in order to induce and/or maintain a mild to moderate sedation, which may be assessed by the MOAA/S and categorized by the following scheme:

TABLE 2

| Level of sedation | MOAA/S score |
|---|---|
| Fully alert | 5 |
| Mild sedation | 4 |
| Moderate sedation | 2-3 |
| Deep sedation | 0-1 |
| Loss of consciousness | 0 |

In one embodiment the sedation profile of the present disclosure will be characterized by: MOAA/S≤4 at three consecutive measurements, e.g. taken every minute; no requirement for a further sedative (e.g. a rescue sedative); and no manual or mechanical ventilation

Hence in one embodiment the present disclosure relates to the use of ubiquinone-5 in combination with an opioid (e.g. fentanyl) without a mechanical or manual ventilation of the patient. Nevertheless supplemental oxygen supply is possible.

In a further aspect of the present disclosure ubiquinone-5 will be administered as a fixed dose per patient. In another aspect of the present disclosure ubiquinone-5 is given to adult patients, i.e. which are 18 years or older.

The compound ubiquinone-5 can be used as free base form or as a pharmaceutically acceptable salt. As salt the besylate or esylate salt of ubiquinone-5 can be used.

According to the present disclosure the ubiquinone-5 will be administered as an intravenous (IV) bolus application, as IV bolus of less than 1 minute, less than 30 or of approximately 15 seconds, which is equivalent to a manual application of an intravenous drug.

In one aspect of the present disclosure the opioid drug will be selected from the group consisting of: morphine, codeine, thebain, papaverin, narcotine, heroin, hydromorphone, dihydrocodeine, thebacon, hydrocodone, oxymorphone, oxycodone, ketobemidone, pethidine, anileridine, piminodine, phenoperidine, furethidine, [alpha]-prodin, trimeperidine, meptazinol, profadol, methadone, dextromoramide, levomethadyl acetate, phenadoxone, dipipanone, themalon, dextropropoxyphene, N-methylmorphinan, levorphanol, dextrometorphane, butorphanol, pentazocine, phenazocine, ketocyclazocine, bremazocine, sufentanil, carfentanil, fentanyl, lofentanil, alfentanil, ohmefentanil, remifentanil, pitramide, benztriamide, diphenoxylate, loperamide, tramadol, tilidine, U-50488, 1-benzyl-4-(4-bromo-phenyl)-4-dimethylamino-cyclohexanol; alfentanil, buprenorphine, butorphanol, codeine, dextromoramide, dextropropoxyphene, dezocine, diamorphine, dihydrocodeine, diphenoxylate, ethylmorphine, etorphine, hydrocodone, hydromorphone, ketobemidone, levomethadone, levomethadyl-acetate, levorphanol, meptazinol, morphine, nalbuphine, nalorphine, oxycodone, oxymorphone, pentazocine, pethidine, piritramide, remifentanil, sufentanil, tilidine, tramadol, tapentadol, met-enkephalin, leu-enkephalin, nociceptin, β-endorphin, endomorphin-1, endomorphin-2, metorphamid, dynorphin-A, dynorphin-B, or a-neoendorphin.

The analgesic drug can be administered as an intravenous bolus application.

In an embodiment, 100 mcg of fentanyl will be given immediately before or together with the initial fixed dose of ubiquinone-5.

In a further aspect of the present disclosure the fentanyl will be administered about 10 minutes before administration of ubiquinone-5, within at least 5 minutes prior to ubiquinone-5 administration, within at least 3 minutes prior to ubiquinone-5 administration or together with ubiquinone-5.

The short time interval between fentanyl dosing and ubiquinone-5 results in a maximum analgesic coverage at the start of the diagnostic or therapeutic intervention. This is important for procedures that start with painful interventions. As an example in colonoscopy the insertion of the scope and its movement through the sigmoid curve of the colon at the beginning is the most inconvenient and painful part of the procedure.

In one aspect of the present disclosure at least one additional (top-up) dose of fentanyl will be given, in the range of 10 to 100 mcg/patient, in the range of 10 to 75 mcg/patient or 25 mcg/patient.

In a further aspect of the present disclosure the time interval between the first fentanyl dose and the top-up dose and/or between two top-up doses will be in the range between 2 to 10 minutes.

In another aspect of the present disclosure ubiquinone-5 will be used for preoperative sedation, amnestic use for perioperative events, or conscious sedation during short diagnostic, operative or endoscopic procedures. In another aspect of the present disclosure ubiquinone-5 will be used for short procedures such as limb resetting or wound dressing. In another aspect of the present disclosure ubiquinone-5 will be used for analgosedation. In another aspect of the present disclosure the use ubiquinone-5 will be contraindicated for subjects with known hypersensitivity to benzodiazepines and subjects with acute narrow-angle glaucoma. ubiquinone-5 may be used in patients with open-angle glaucoma only if they are receiving appropriate therapy.

In a further aspect of the present disclosure the pharmacological effect ubiquinone-5 and/or the opioid drug will be reversed by another drug, which is referred to as a “reversal agent”.

As reversal drug for the ubiquinone-5, a GABA receptor antagonist will be used, which can be flumenazil.

As reversal drug for the opioid drug an opioid receptor antagonist will be used, such as naloxone.

In some embodiments, the present disclosure is directed to a method for conducting a procedure involving sedation in a subject comprising: (a) administering intravenously to the subject one or more fixed doses of a pharmaceutical composition in an amount sufficient to sedate the subject to induce moderate anesthesia, wherein the pharmaceutical composition comprises ubiquinone-5 or a pharmaceutically acceptable salt thereof; and (b) passing an endoscope into the subject.

In some embodiments, the subject will be 18 years or older. In some embodiments, one or more doses of an opioid will be administered to the subject prior to the administration of the pharmaceutical composition to the subject. In some embodiments, the opioid will be fentanyl. In some embodiments, the procedure will be an upper GI endoscopy. In some embodiments, the procedure will be a colonoscopy.

In some embodiments, one or more fixed doses of the pharmaceutical composition will be administered to the subject over a time period of one minute or less. In some embodiments, each fixed dose of the pharmaceutical composition administered to the subject will comprise from about 2 mg to about 10 mg of the ubiquinone-5, or a pharmaceutically acceptable salt thereof. In some embodiments, each fixed dose of the pharmaceutical composition administered to the subject will comprise from about 3 mg to about 10 mg of the ubiquinone-5, or a pharmaceutically acceptable salt thereof. In some embodiments, each fixed dose of the pharmaceutical composition administered to the subject will comprise from about 3 mg to about 9 mg of the ubiquinone-5, or a pharmaceutically acceptable salt thereof. In some embodiments, each fixed dose of the pharmaceutical composition administered to the subject will comprise about 5 mg of the ubiquinone-5. In some embodiments, the one or more doses of the pharmaceutical composition will comprise an amount of the ubiquinone-5, or a pharmaceutically acceptable salt thereof, to achieve an MOAA/S score of less than or equal to 4 in the subject. In some embodiments, the pharmaceutical composition will comprise the besylate salt of the ubiquinone-5.

In one or more embodiments of the present disclosure, CoQ2 will be used instead of uniquinone-5 in any of the above described compositions, methods of use thereof, or methods to prepare said composition. The CoQ2 composition will be an anesthetic, sedative agent, hypnotic agent, or combination thereof.

In one or more embodiments of the present disclosure, CoQ3, CoQ4, or CoQ5 will be used instead of uniquinone-5 in any of the above described compositions, methods of use thereof, or methods to prepare said composition. The CoQ3, CoQ4, or CoQ5 composition will be an anesthetic, sedative agent, hypnotic agent, or combination thereof.

In one or more embodiments of the present disclosure, CoQ6, CoQ7, or CoQ8 will be used instead of uniquinone-5 in any of the above described compositions, methods of use thereof, or methods to prepare said composition. The CoQ6, CoQ7, or CoQ8 composition will be an anesthetic, sedative agent, hypnotic agent, or combination thereof.

### Example 1

As an example, the present composition containing ubiquinone-5 or its pharmaceutically acceptable salts thereof was formulated as follows.

TABLE 3

| | UB5 Calculation |
|---|---|
| UB5 stock mass (mg) | 10 |
| Ethanol desired conc. | 5% |
| UB5 desired conc. (mg/ml) | 20 |
| UB5 (mg) | 10 |
| Total Volume (μl) | 500 |
| Ethanol Volume (μl) | 25 |
| Intralipid Volume (μl) | 475 |

The intralipid is 20% which is phospholipid stabilized soybean oil. It contains 20% soybean oil, 1.2% egg yolk phospholipids, 2.25% glycerin, and water.

Without wishing to be bound by theory, it is proposed that ubiquinone-5 causes an increase in mitochondrial leak and precipitates a decline in mitochondrial membrane potential (ΔΨm) during in vitro exposure. Oxidation of substrates coupled with proton pumping within the electron transport chain (ETC) is the process that generates ΔΨm, while proton leak and ATP turnover consume it.

### Example 2

A composition containing ubiquinone-5 and decylubiquinone or its pharmaceutically acceptable salts thereof will be formulated. The make-up of the composition will be about 2% (w/v) ubiquinone-5; about 2% (w/v) decylubiquinone; about 5% (v/v) ethanol; about 19% (v/v) soybean oil; about 2.1375% (v/v) glycerin; about 1.14% (v/v) egg-yolk phospholipid; and about 72.7225% water.

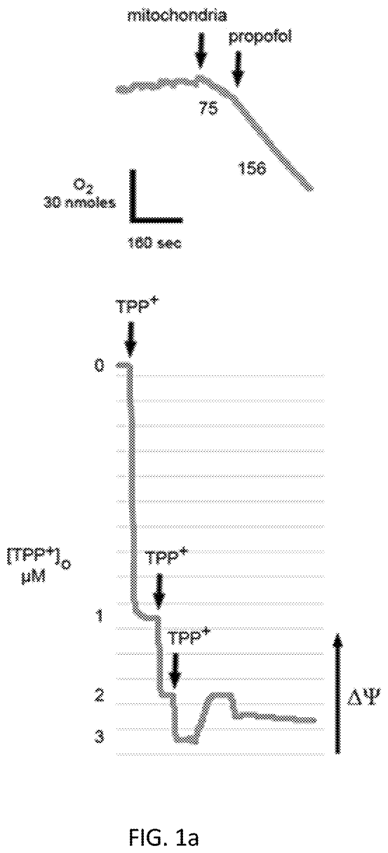

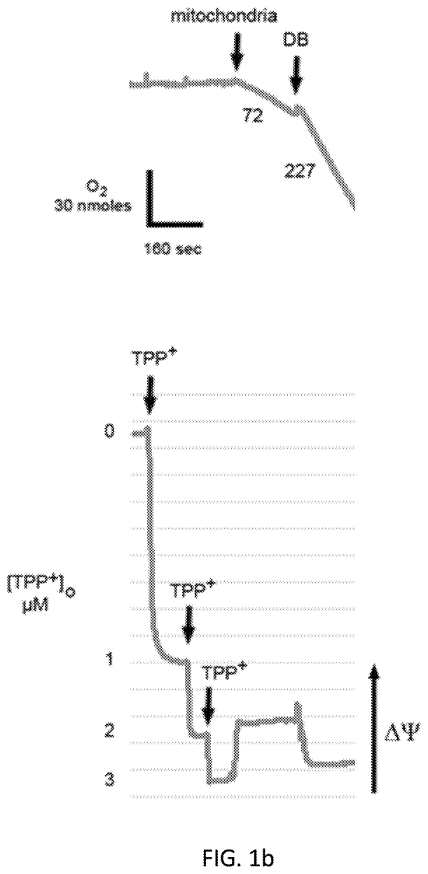

FIG. 1 shows that Ub5 induced excessive proton leak and caused a precipitous decline in ΔΨm. In FIG. 1, representative tracings of Complex II-dependent state 4 respiration (leak respiration) using succinate in the presence of oligomycin are depicted in the top three curves and tracings of simultaneously measured mitochondrial membrane potential (ΔΨ) are shown in the bottom three curves. The numbers in the tracings above show rates of oxygen consumption (nmol·mL-1·min-1·mg mitochondrial protein-1). Addition of propofol (FIG. 1 a) decylubiquinone (DB) (FIG. 1b) or ubiquinone-5 (FIG. 1c) are indicated. A was determined as a function of triphenylphosphonium (TPP+) concentration in the bath. Both quinone analogs increased proton leak as evidenced by a concomitant increase in state 4 respiration and decline in AY. This mechanism is highly reminiscent of the effect of the anesthetic, propofol (FIG. 1a), in isolated mitochondria.

The mitochondrial membrane potential (ΔΨm), the major component of the proton motive force, is required for ATP synthase to produce ATP via oxidative phosphorylation. Defects in the ability of mitochondria to generate or sustain ΔΨm limit bioenergy availability and jeopardize cellular homeostasis. Neurons rely predominantly on oxidative metabolism for neurotransmission and changes in ΔΨm affect synaptic activity. ΔΨm plays an important overarching role in regulating a number of cellular processes which include ATP synthesis. Although ΔΨm and ATP are often thought to be inextricably linked, they differ with regard to cellular signaling. In bacteria, for example, changes in ΔΨm affect motility independent of ATP while, in yeast, it is the loss of A′m, not ATP, that triggers responses to extend lifespan. This ΔΨm may, in fact, be more important to maintaining cellular homeostasis than ATP. Without wishing to be bound by theory, it is proposed that anesthetics induce unconsciousness by causing a precipitous fall in ΔΨm within neuronal mitochondria. It appears that Ub5 induces excessive proton leak coupled with inhibition of electron transfer to inhibit and/or prevent mitochondria from generating and maintaining an adequate A′m.

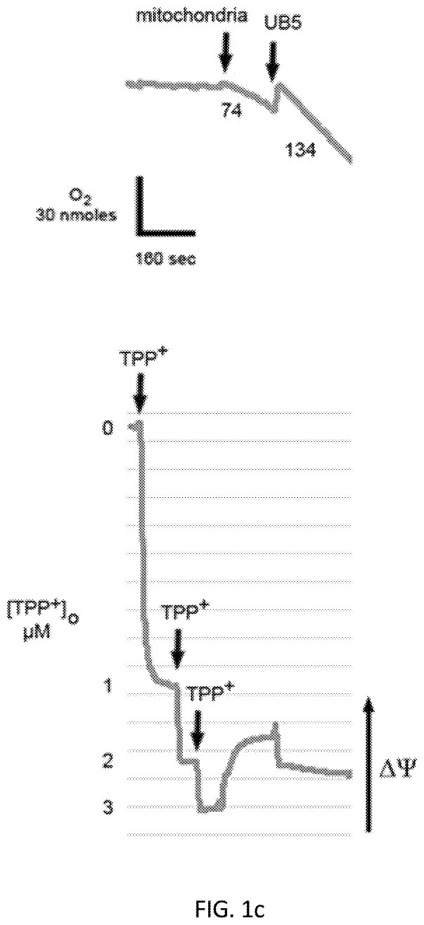

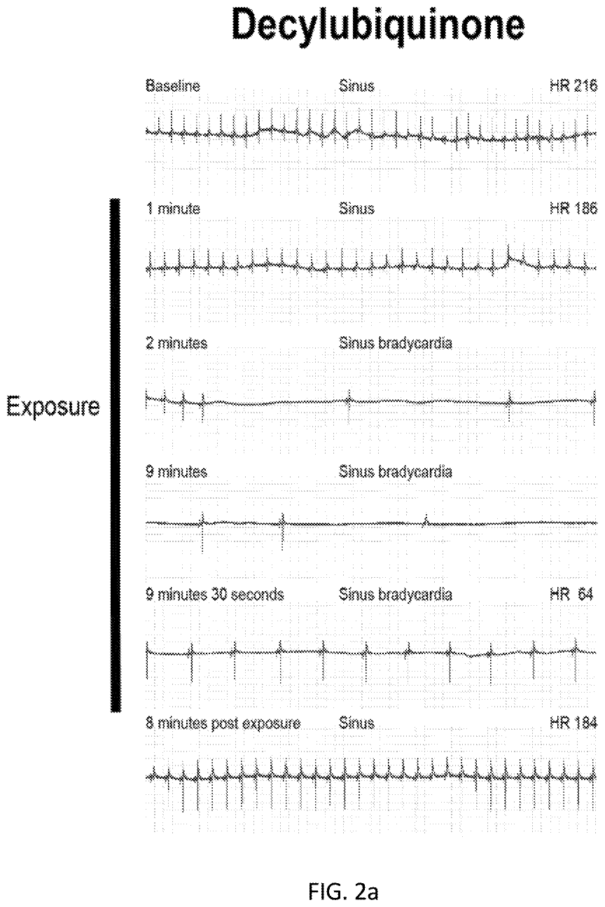

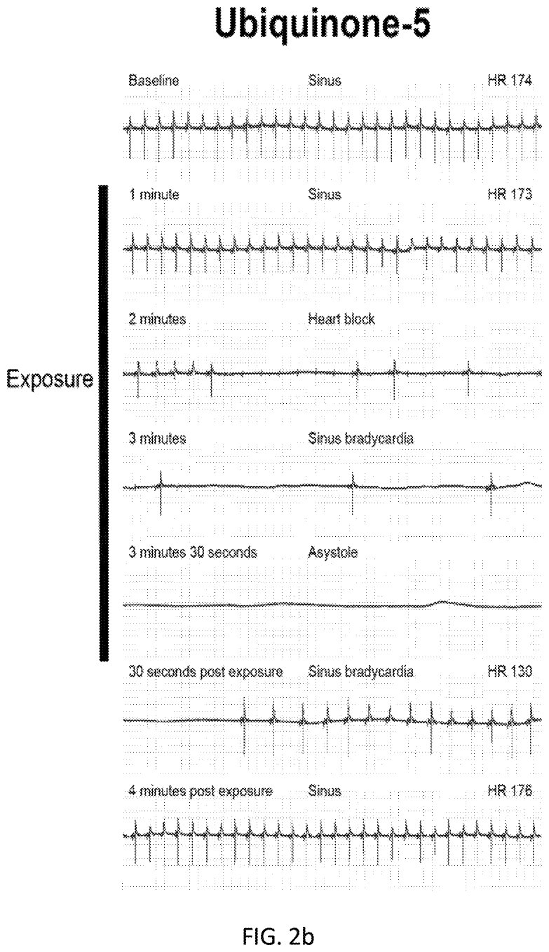

In FIGS. 2a and 2b, surface ECGs were recorded continuously for isolated perfused murine hearts that were prepared and stabilized. Hearts were exposed to either decylubiquinone or ubiquione-5. Decylubiquinone rapidly induced bradycardia that persisted throughout the duration of exposure. Upon cessation of exposure and washout, sinus rhythm normalized. Ubiquinone-5 rapidly induced heart block and bradycardia that progressed to asystole and electrical silence that persisted throughout the duration of exposure. Upon cessation of exposure and washout, sinus rhythm normalized. Thus, quinone analogs suppressed electrical signaling in the heart in a reversible manner.

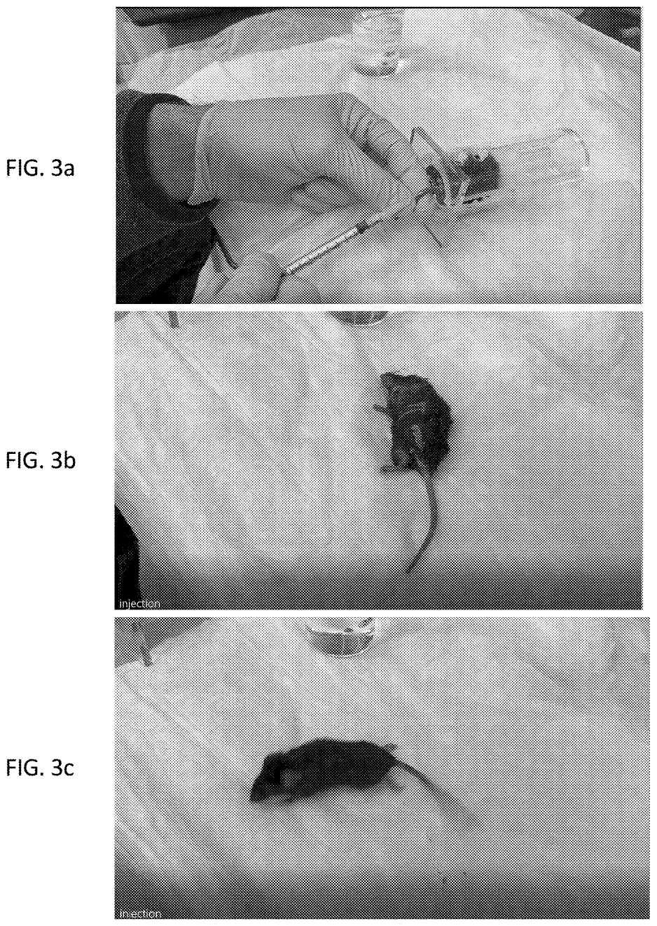

FIGS. 3a-3c show that ubiquinone-5 induces short-term hypnosis immediately following tail vein injection. FIG. 3a shows an awake mouse undergoing tail vein injection with UB5 (200 mg/kg). FIG. 3b shows the loss of righting reflex immediately after injection. FIG. 3c shows that the mouse regains righting reflex within 3-4 minutes of injection.

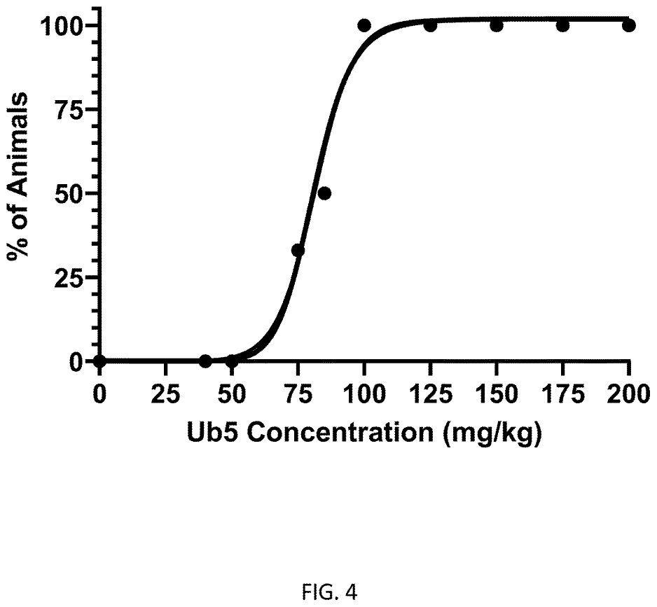

FIG. 4 shows a ubiquinone-5 dose-response curve. 50 C57Bl/6 mice were injected with various doses of ubiquinone-5 via tail vein. Loss of righting reflex (LORR) was determined and percentage of mice injected was calculated. The ED₅₀ was 81.3 mg/kg (95% CI 80.3-82.4). R²=0.99.

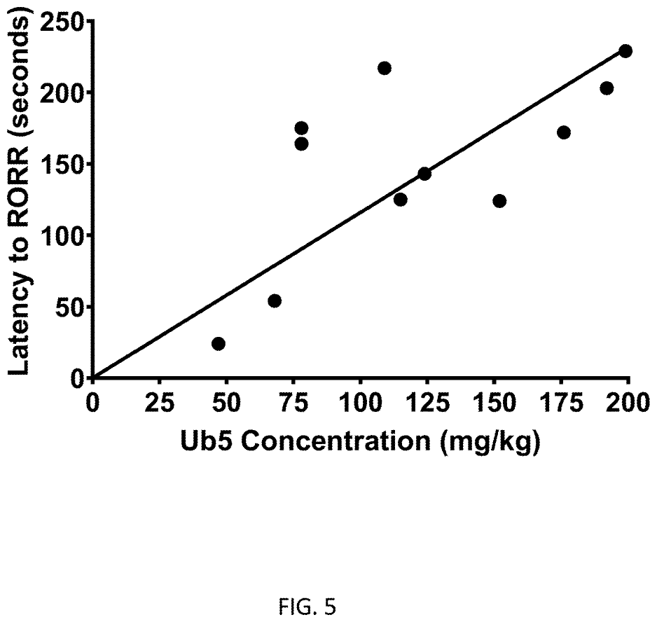

FIG. 5 shows the latency to return of righting reflex for mice injected with Ub5 as a function of the concentration of the injection. The time to RORR correlated significantly with injected dose. P<0.001, R²=0.52. N=13 mice.

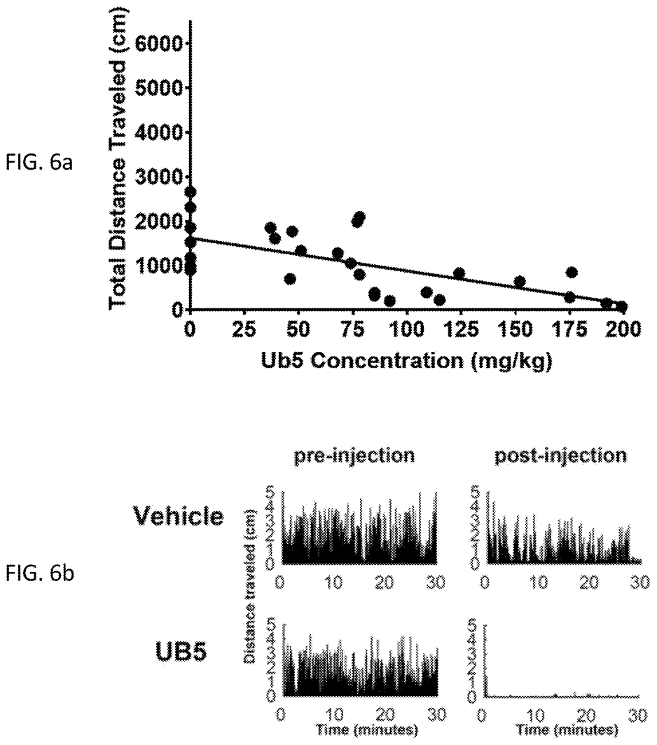

Representative mice, one with a tail vein Ub5 injection (200 mg/kg) and in 1 vehicle-injected mouse were placed in a circular open field (radius 76 cm) and ambulation was recorded for 30 minutes prior to injection using a camera mounted above. Following tail vein injection and return of righting, the mice were placed in a novel circular open field and ambulation was recorded for a subsequent 30 minutes. Total distance traveled was quantified and differences between pre- and post-injection was assessed and compared within and between groups. FIG. 6a shows the total distanced traveled as a function of Ub5 concentration injection. There was a significant negative correlation with Ub5 dose. P<0.001, R²=0.47. N=31 mice. FIG. 6b shows representative data depicting distance traveled within the open field every 200 ms for 30 minutes pre- and post-injection.

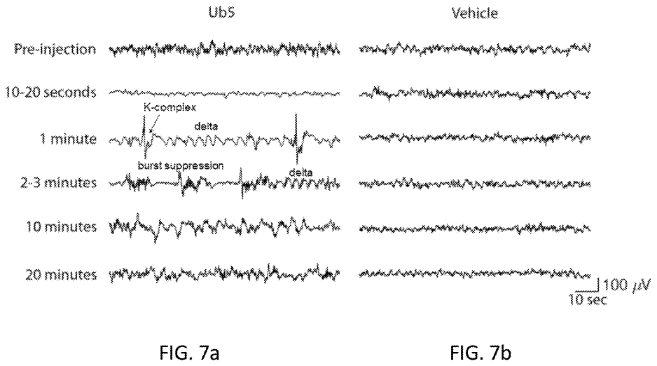

FIGS. 7a and 7b shows EEG signals for mice that were injected with either Ub5 (200 mg/kg) or intralipid vehicle via tail vein before, during, and post-injection. Mice had indwelling epidural EEG leads (digitized at 1000 Hz, 6 leads, 32 channel headstages, previously implanted 2 weeks prior). Representative EEG tracings obtained prior to injection and post-injection (up to 20 minutes) are depicted. High frequency activity is seen pre-injection in both mice and in vehicle-injected mouse throughout. Low amplitude activity occurs 10-20 seconds post-Ub5 injection. At 1 minute, delta waves, K-complexes, and spindles are seen. Between 2-3 minutes, burst suppression and delta waves appear. Delta waves predominate at 10 minutes post-Ub5 injection and high frequency activity is restored by 20 minutes. The slow-wave EEG activity seen following injection confirmed Ub5-induced hypnosis while burst suppression provided evidence of a deep state of anesthesia. Ub5 was observed to shift the power in the spectrum to lower frequency bandwidths.

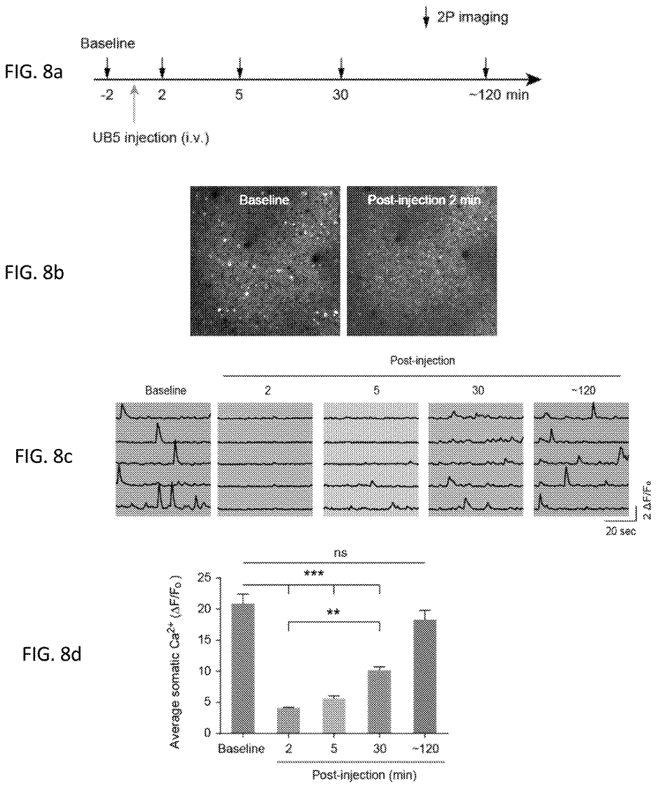

FIG. 8a shows a time line for imaging of neurons within layer II/III of the somatosensory cortex in awake Thy1.2-GCaMP6 transgenic mice through a cranial window using a two-photon microscope. Time-lapsed images were obtained prior to injection and for 30 minutes post-tail vein Ub5 injection and neuronal GCaMP6 (genetically encoded calcium indicator) fluorescence was quantified. FIG. 8b shows that fluorescent Ca²⁺ transients were easily seen within neuronal somata at 25X. FIG. 8c shows fluorescent traces of Ca²⁺ transients observed from 60-100 neurons over time. FIG. 8d graphs the quantification of mean Ca²⁺ fluorescence. Ub5 markedly decreased Ca²⁺ transients, indicating a decrease in neuronal activity. **p<0.01, ***p<0.001.

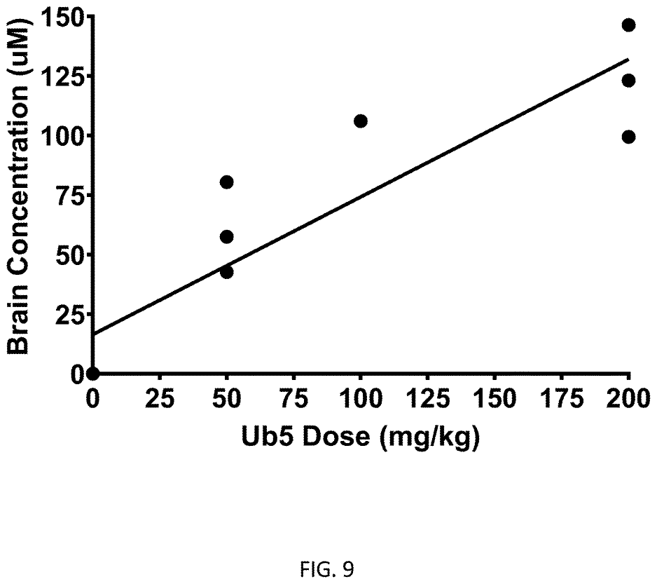

FIG. 9 shows data for the concentration of Ub5 in the brain following tail vein injection. Using an LC-MS approach, the concentration of Ub5 in brain extracts harvested immediately following intravenous injection (upon onset of loss of righting) was quantified. Injection of 200 mg/kg Ub5 resulted in a tissue concentration of ~100 μM.

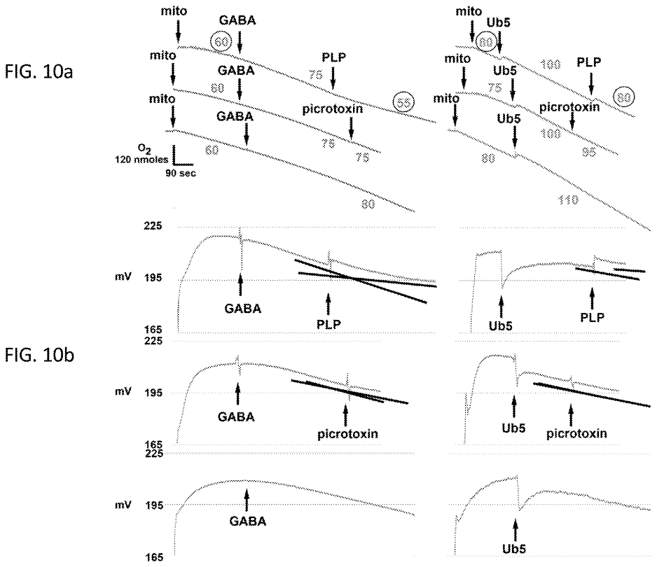

Turning to FIGS. 10a and b, oligomycin-induced state 4 was initiated in isolated forebrain mitochondria using succinate. Tracings of 02 consumption (FIG. 10a) with simultaneous ΔΨm measurement (FIG. 10b) are depicted. Numbers (FIG. 10a) are rates of respiration (nmol·mL-1·min-1·mg mitochondrial protein-1). GABA (10 mM) or Ub5 (100 mM) were added. Aralar was inhibited with pyridoxal 5′-phosphate (PLP)(10 mM). Picrotoxin (GABAA receptor antagonist (1 mM)) and GABA or Ub5 alone served as controls. Black solid lines are slopes of ΔΨm prior to and after inhibitor. Decrease in respiration rate with concomitant stabilization or rise in ΔΨm indicates inhibition of leak. GABA induced a slow increase in proton leak (increased respiration with a gradual fall in ΔΨm) while Ub5 induced leak immediately (sudden increase in respiration with a precipitous decline in ΔΨm). Picrotoxin had minimal effect. PLP normalized respiration to pre-GABA or Ub5 levels (circled numbers) and stabilized ΔΨm. Thus, PLP blocked GABA import via Aralar. With Ub5, PLP acutely increased ΔΨm. Normalization of respiration to pre-Ub5 levels with a concomitant rise and stabilization of ΔΨm indicates that Aralar is a major source of Ub5-induced leak. Aralar is best described as an aspartate-glutamate carrier within mitochondria. Aralar is the solute that exports aspartate from neuronal mitochondria in exchange for glutamate plus a proton. Thus, Aralar causes leak by translocating protons across the inner mitochondrial membrane during solute transport. Aralar causes leak that can degrade ΔΨm. Aralar is a neuronal transporter that sequesters GABA within the mitochondrial matrix. Many anesthetic agents bind to the GABAA receptor and potentiate GABAergic signaling.

The present disclosure shows that tail vein injection of the short-chain CoQ analog, ubiquinone-5 immediately induced unconsciousness in mice. Ub5-induced hypnosis was short-lived and mice regained consciousness within minutes.

The present disclosure has been described above and in the attached figures; modifications and variations will be apparent to those of ordinary skill in the art from this description and the scope of protection is to be determined by the claims that follow.

1. A pharmaceutical composition comprising a coenzyme Q2 (CoQ2) containing oil-in-water emulsion.

2. The composition of claim 1, wherein CoQ2 is stabilized by a surfactant.

3. The composition of claim 1, wherein the CoQ2 is dissolved in a water-immiscible solvent.

4. The composition of claim 1, wherein CoQ2 is emulsified with water.

5. The composition of claim 1, further comprising an excipient selected from the group consisting of an amino acid, a vitamin, a mineral, and combinations thereof.

6. The composition of claim 1, wherein CoQ2 is present in an amount ranging from about 0.01% (w/v) to 5% (w/v).

7. The composition of claim 1, wherein the pH of the composition ranges from about 5.0 to about 8.0.

8. The composition of claim 1, further comprising a tonicity modifier.

9. The composition of claim 8, wherein the composition is isotonic with blood.

10. The composition of claim 1, further comprising ascorbic acid or its pharmaceutically acceptable salt thereof.

11. The composition of claim 1, wherein the composition is for administration by a route selected from the group consisting of intravenous, inhalational, subcutaneous, intramuscular, transdermal, and parenteral administration.

12. The composition of claim 1, further comprising decylubiquinone.

13. The composition of claim 12, wherein the ratio by weight of ubiquinone-5 to decylubiquinone is 1:1, 2:1, 3:1, 4:1, 5:1, 1:2, 1:3, 1:4, or 1:5.

14. A method of inducing anesthesia in a subject, the method comprising administering to the subject an effective amount of a composition comprising a CoQ2-containing oil-in-water emulsion.

15. The method of claim 14, wherein the composition is administered by a single bolus injection or a number of bolus injections that ranges from 2 to 10.

16. The method of claim 14, wherein the composition is administered by continuous infusion.

17. The method of claim 14, wherein the composition is administered at a dose sufficient to achieve a desired clinical anesthetic endpoint selected from the group consisting of general anesthesia, moderate sedation, tranquilization, immobility, amnesia, analgesia, deep sedation and autonomic quiescence.

18. The method claim 14, wherein the composition is administered by a route selected from the group consisting of intravenous, inhalational, subcutaneous, intramuscular, transdermal, and parenteral.
